(19) United States
(12) Plant Patent Application Publication
(10) Pub. No.: US 2012/0030848 P1
(43) Pub. Date: Feb. 2, 2012

(54) Salvia plant named 'DANSALFN13'

(50) ;

(75) Inventor: Gavriel Danziger, Moshav Nir-Zvi (IL)

(73) Assignee: DANZIGER "DAN" FLOWER FARM

(21) Appl. No.: 12/805,426

(22) Filed: Jul. 30, 2010

(51) Int. Cl. A01H 5/00

(52) U.S. Cl. PLT/475

(57) ABSTRACT

A new and distinct Salvia farinacea plant named ‘DANSALFN13’ particularly characterized by bilabiate-shaped flowers with petals in shades of white; upright and compact growth habit; freely branching and flowering plant habit; about 6 to 8 weeks flowering response and stays in full bloom in moderate climate; and good weather tolerance with a preferred temperature range of 5° C. to 35° C.

### LATIN NAME OF THE GENUS AND SPECIES OF THE PLANT CLAIMED

Salvia farinacea

### VARIETY DENOMINATION

‘DANSALFN13 ’

### BACKGROUND OF THE INVENTION

The present invention relates to a new and distinct cultivar of Salvia plant, botanically known as Salvia farinacea of the Lamiaceae family, and hereinafter referred to by the variety denomination ‘DANSALFN13’

The new Salvia farinacea ‘DANSALFN13’ is a product of a planned breeding program conducted by the inventor, Gavriel Danziger, in Moshav Mishmar Hashiva, Israel. The objective of the breeding program was to develop a new Salvia farinacea cultivar with compact and well branched habit with white flower color.

The new Salvia farinacea ‘DANSALFN13’ originated in 2007 as a result of open pollination between two unknown, unpatented, Salvia farinacea plants growing in a field in the controlled breeding program conducted by the inventor, Gavriel Danziger, in Moshav Mishmar Hashiva, Israel.

The new Salvia farinacea ‘DANSALFN13’ was discovered and selected by the inventor, Gavriel Danziger, as a single flowering plant within the progeny of the stated open pollination cross in a controlled environment in 2007 in Moshav Mishmar Hashiva, Israel.

Asexual reproduction of the new Salvia farinacea ‘DANSALFN13’ by vegetative cuttings was first performed in August of 2008 in Moshav Mishmar Hashiva, Israel, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘DANSALFN13’ which in combination distinguish this Salvia farinacea as a new and distinct cultivar:

1. Plants produce bilabiate-shaped flowers with petals in shades of white;

2. Upright and compact growth habit;

3. Freely branching and flowering plant habit;

4. About 6 to 8 weeks flowering response and stays in full bloom in moderate climate; and

5. Good weather tolerance with a preferred temperature range of 5° C. to 35° C.

The new Salvia farinacea ‘DANSALFN13’ is an open pollination product and therefore, can not be compared to its parents.

Of the many commercial cultivars known to the present inventor, the most similar in comparison to the new Salvia farinacea ‘DANSALFN13’ is the Salvia farinacea ‘DANSALFUN1’ (patented, U.S. Plant Pat. No. 19,788), also know by the tradename of Salvia Sallyfun Blue. Plants of ‘DANSALFN13’ differ from plants of the ‘DANSALFUN1’ in the characteristics described in Table 1:

TABLE 1

| | | Comparison Cultivar |
|---|---|---|
| | New Cultivar | ‘DANSALFUN1’ |
| Trait | ‘DANSALFN13” | (patented) |
| Mature Flower | Petal shades of | Petal shades of violet- |
| Color (upper | white, closest to | blue, closest to |
| surface) | RHS N999D | RHS 94A |
| Inflorescence | Height: About | Height: About |
| size | 10 cm to 15 cm | 15 cm to 18 cm |
| | Height: About 0.5 cm | Diameter: About 1.5 cm |
| Flower size | Depth: About 0.8 cm | Depth: About 0.7 cm |
| (lower lip) | Width: About 1.0 cm | Width: About 1.0 cm |

### BRIEF DESCRIPTION OF THE PHOTOGRAPHS

The accompanying photographs illustrate the overall appearance of the new Salvia DANSALFN13′ showing the colors as true as is reasonably possible with colored reproductions of this type. Colors in the photographs may differ slightly from the color values cited in the detailed botanical description, which accurately describe the color of ‘DANSALFN13’.

FIG. 1 shows a side perspective view of a typical flowering plant of ‘DANSALFN13’ in a 12 cm, potted at 2 months of age.

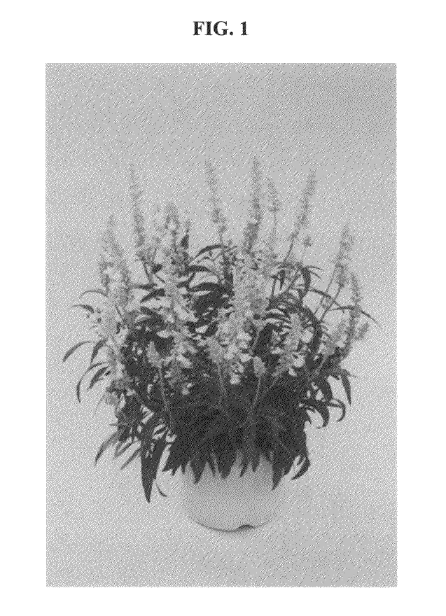

FIG. 2 shows a close-up of the typical flowers, buds and leaves of ‘DANSALFN13’, potted at 2 months of age.

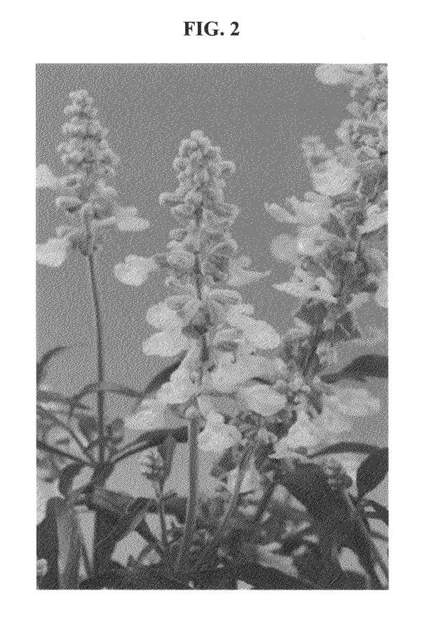

### DETAILED BOTANICAL DESCRIPTION

The new Salvia ‘DANSALFN13’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘DANSALFN13’ as grown in a greenhouse in Moshav Mishmar Hashiva, Israel, under conditions which closely approximate those generally used in commercial practice. Optimal growth conditions include that plants of ‘DANSALFN13’ are grown outdoors in a moderate climate and indoors during the winter season. Irrigation and fertilization are recommended on a regular basis. Plants of ‘DANSALFN13’ are usually grown in full sunlight. Plants of ‘DANSALFN13’ are fully grown in 1½ months from planting a cutting in the summer season and bloom all year-round in a moderate climate. The ‘DANSALFN13’ plants described herein were grown indoors in 12 cm pots in a glass greenhouse in Moshav Mishmar Hashiva, Israel, with the day temperatures ranging from 18° C. to 28° C. and night temperatures ranging from 11° C. to 18° C. Plants of ‘DANSALFN13’ were fertilized up to a level of 250-300 ppm N. No growth retardant(s) are used.

Color references are made to The Royal Horticultural Society Colour Chart (R.H.S.), 2001 edition, except where general colors of ordinary significance are used. Color values were taken under daylight conditions at approximately noon in Moshav Mishmar Hashiva, Israel. The age of the ‘DANSALFN13’ plants described is 8 weeks.
Botanical: Salvia farinacea
Parentage:
   Seed parent.—Unknown, unpatented Salvia farinacea cultivar.
   Pollen parent.—Open pollination.
Propagation:
   Type.—Vegetative cuttings.
   Rooting habit and description.—Fine, fibrous and freely branching.
   Time to initiate roots.—About 5 to 8 days at 25° C.
   Time to produce a rooted cutting.—About 21 days at 25° C.
Plant:
   Form.—Upright and erect.
   Growth habit.—Compact.
   Height (from soil level to top of plant).—About 30 cm to 40 cm.
   Spread.—About 20 cm to 30 cm.
   Cold tolerance.—Frost tender. Temperatures below 5° C. may damage plants.
   Vigor.—Vigorous growth; plants are fully grown in 1.5 months from planting cutting in during summer season.
Lateral branch:
   Quantity per plant.—About 35 to 40.
   Strength.—Strong.
   Length.—About 14 cm.
   Diameter.—About 0.3 cm.
   Aspect.—Upright.
   Texture.—Glabrous with very delicate short feathers like hairs.
   Color.—Yellow Green group, RHS 144A.
   Internode length.—About 1.5 cm to 2 cm.
   Internode color.—Yellow Green group, RHS 144A.
Foliage:
   Quantity of leaves per lateral branch.—About 10 to 12.
   Type.—Simple.
   Arrangement.—Opposite.
   Length.—About 6 cm.
   Width.—About 2 cm.
   Overall shape of leaf.—Lance-shaped.
   Apex.—Acute.
   Base.—Attenuate.
   Margin.—Very delicate, serrate.
   Texture.—Upper surface: Glabrous Under surface: Slightly pubescent.
   Color of upper surface.—Mature leaf: Green, RHS 137C Immature leaf: Yellow-green, RHS 146B.
   Color of under surface.—Mature leaf: Green, RHS 137B Immature leaf: Yellow-green, RHS 146B.
   Fragrance.—Very delicate, sage-like fragrance.
   Venation.—Pattern: Pinnate. Color: Upper surface: Yellow-green, RHS147D Under surface: Yellow-green, RHS 146C.
   Petioles.—Length: About 0.6 cm to 0.8 cm Diameter: About 1.0 mm Texture: Upper surface: Glabrous with very delicate short feathers like hairs; Under surface: Glabrous with very delicate short feathers like hairs.
   Color.—Green, RHS 137B.
Flowering:
   Blooming period.—All year round in a moderate climate or in a greenhouse environment. In Israel, flowering occurs between February and November outdoors.
   Flowering response time.—About 6 weeks from planting of unrooted cutting.
   Flowering longevity.—On the plant: about 3 to 5 days, depending on temperature; flowers persistent.
Inflorescence:
   Arrangement and type.—Bilabiate flowers arranged in verticillasters on spikes.
   Orientation at opening.—Facing outward.
   Quantity.—Per Plant with at least one open flower: About 10 (spikes) at 8 weeks. Per Verticillaster with fully open flowers: About 6.
   Inflorescence size.—Height: About 10 cm to 15 cm Diameter: About 0.5 cm.
   Flower size.—Length: Upper lip: about 0.5 cm; Lower lip: about 0.8 cm Depth: Upper lip: about 0.3 cm; lower lip: about 1 cm.
   Fragrance.—Very sweet, nectar-like fragrance.
Bud:
   Rate of opening.—About 1 to 2 days, according to weather.
   Shape.—Ovoid.
   Length.—About 6 mm.
   Diameter.—About 3 mm.
   Color.—Apex: Green-white, RHS N157B Base: Green-white, RHS N157B.
   Texture (both surfaces).—Lightly pubescent with very delicate short feathers like hairs.
Petals:
   Quantity.—Two.
   Arrangement.—One upper lip and one lower lip with 3 lobes. Lips are fused at base. Upper lip has smooth short feather like hairs, lower lip is smooth and hairless.
   Upper lip.—Overall shape: Oblong. Apex shape: Rounded Base shape: Fused Length (from throat): About 1.5 cm Width: About 1.0 cm Margin: Entire Texture: Upper surface: Smooth, short feather like hairs; Under surface: Glabrous. Color: When opening: Upper surface (including margin): Green-white, RHS 157B; Under surface (including margin): White, RHS N999D. When fully opened: Upper surface (including margin): Green-white, RHS 157B; Under surface (including margin): White, RHS N999D.
   Lower lip.—Overall shape: Rounded Apex shape: Blunt Base shape: Fused Length: Lateral lobe: About 0.7 cm; Central lobe: About 1.0 cm. From throat: About 1.6 cm (please confirm) Width: Lateral lobe: About 0.4 cm; Central lobe: About 0.8 cm. Margin: Entire.
   Texture.—Upper lip: Upper surface: Smooth, short feather like hairs; Under surface: Glabrous; Lower lip: Upper surface: Glabrous; Under surface: Glabrous.
   Color.—When opening: Upper surface (including margin): Green-white, RHS 157B; Under surface (including margin): White, RHS N999D. When fully opened: Upper surface (including margin): Green-white, RHS 157B; Under surface (including margin): White, RHS N999D.
Throat:
   Length.—About 0.3 cm.
   Width.—About 0.3 cm.
   Texture.—Outside: Glabrous; Inside: Glabrous.
   Color.—Outside: White, RHS N999D Inside: White, RHS N999D.
Calyx:
   Shape.—Two sepals fused into a tube.
   Length.—About 2 mm.
   Diameter.—About 2 mm.
Sepals:
   Quantity per flower.—2, fused to form a tube.
   Length.—About 0.7 cm.
   Width.—About 0.4 cm.
   Shape.—Tubular.
   Apex.—Mucronulate.
   Base.—Fused.
   Margin.—Entire.
   Texture.—Upper: Glabrous, with rough lines; Under: Glabrous.
   Color.—Upper: Green-white, RHS N157B Under: Yellow-green, RHS 144A.
Peduncle:
   Length.—About 4.0 cm.
   Diameter.—About 0.5 mm.
   Angle.—About 30° from vertical.
   Strength.—Strong.
   Texture.—Glabrous, with smooth, short feather-like hairs.
   Color.—Closest to grey-green, RHS 189D.
Pedicels:
   Length.—About 0.5 cm.
   Diameter.—About 1.0 mm.
   Angle.—About 30° from peduncle axis.
   Strength.—Strong.
   Texture.—Glabrous, with smooth, short feather-like hairs.
   Color.—Closest to grey-green, RHS 192D.
Reproductive organs:
   Androecium.—Stamen: Number: 2 per flower. Length: About 5 mm Color: White, RHS N999D Anther: Length: About 1.5 mm Width: About 1.0 mm Color: White, RHS 199D Filament: Length: About 3 mm to 4 mm Color: White, RHS N999D Pollen: Amount: Sparce Color: Closest to yellow-orange, RHS 165A.
   Gynoecium.—Pistil: Number: One per flower Length: About 1.0 mm Stigma: Length: About 3 mm Width: About 0.5 mm Color: White, RHS N999D Style: Length: About 8 mm Color: White, RHS N999D Ovary: Length: About 12 mm Width: About 1.0 mm Color: White, RHS N999D.
Seed:
   Quantity.—About 4.
   Length.—About 2.5 mm.
   Width.—About 1.5 mm.
   Shape.—Ovoid.
   Texture.—Smooth.
   Color.—Greyed-orange, RHS 164B.
Fruit: None
Disease/pest resistance: No information is currently available.
Disease/pest susceptibility: Typically not affected by pests, occasionally drips. Damping-off in seed beds, downy and powdery mildews and botrytis.

What is claimed is:

1. A new and distinct Salvia farinacea plant named ‘DANSALFN13’, as illustrated and described herein.
